(19) United States
(12) Patent Application Publication
(10) Pub. No.: US 2022/0362292 A1
(43) Pub. Date: Nov. 17, 2022

(54) ADOPTIVE TRANSFER OF PLASMACYTOID DENDRITIC CELLS TO PREVENT OR TREAT HAIR LOSS

(71) Applicant: Tufts Medical Center, Inc., Boston, MA (US)

(72) Inventors: Pedram HAMRAH, Wellesley, MA (US); Arsia JAMALI, Boston, MA (US)

(21) Appl. No.: 17/629,918

(22) PCT Filed: Jul. 24, 2020
(86) PCT No.: PCT/US2020/043384

§ 371 Date: Jan. 25, 2022

Related U.S. Application Data
(60) Provisional application No. 62/878,897, filed on Jul. 26, 2019.

(51) Int. Cl. A61K 35/15; A61P 17/14; A61K 31/713; A61K 9/00; A61K 8/49; A61K 31/58

(52) U.S. Cl. CPC A61K 35/15; A61P 17/14; A61K 31/713; A61K 9/0014; A61K 8/4953; A61K 31/58

(57) ABSTRACT

The invention provides methods of preventing or treating hair loss by adoptive transfer of plasmacytoid dendritic cells and related compositions.

### BACKGROUND OF THE INVENTION

Alopecia is defined as the loss of hair from the body. Hair grows in cycles, with each cycle consisting of three phases: anagen (long growing phase), catagen (brief transitional apoptotic phase), and telogen (short resting phase). At the end of the resting phase, the hair falls out (exogen) and a new hair starts growing in the follicle, beginning the cycle again. Normally, about 50 to 100 scalp hairs reach the end of resting phase each day and fall out. When significantly more than 100 hairs/day go into resting phase, clinical hair loss (telogen effluvium) may occur. A disruption of the growing phase causing abnormal loss of anagen hairs is an anagen effluvium.

Alopecia can be categorized as scarring or non-scarring alopecia. Scarring alopecia is the result of active destruction of the hair follicle. In scarring alopecia, the follicle is irreparably damaged and replaced with fibrotic tissue. Non-scarring alopecia results from processes that reduce or slow hair growth without irreparably damaging the hair follicle. Several hair disorders show a biphasic pattern in which non-scarring alopecia occurs early in the course of the disease, and then scarring alopecia and permanent hair loss occurs as the disease progresses. Scarring alopecias can be subdivided further into primary forms, in which the target of inflammation is the follicle itself, and secondary forms, in which the follicle is destroyed as a result of nonspecific inflammation. Disorders that primarily affect the hair shaft (trichodystrophies) are considered non-scarring alopecia.

The most common causes of alopecia include androgenetic alopecia (male-pattern or female-pattern hair loss), which is an androgen-dependent hereditary disorder that may eventually affect up to 80% of white men by the age of 70 (male-pattern hair loss) and about half of all women (female-pattern hair loss); drugs (including chemotherapeutic agents); infection (e.g., tinea capitis and kerion); systemic disorders (e.g., disorders that cause high fever, systemic lupus erythematosus, endocrine disorders, and nutritional deficiencies); alopecia areata; trauma (including trichotillomania, traction alopecia, central centrifugal cicatricial alopecia, burns, radiation, and pressure-induced (e.g., postoperative) hair loss); primary hair shaft abnormalities; autoimmune diseases; heavy metal poisoning; and rare dermatologic conditions (e.g., dissecting cellulitis of the scalp).

Forty percent of males demonstrate noticeable hair loss by age 35, 65% by age 60, and 80% by age 80. Alopecia affects approximately 50% of females. The number of hair loss sufferers, world-wide, seeking professional treatment more than doubled between 2004 and 2008, from 361,077 to 811,363.

While currently there are medications, including the potassium channel openers (e.g., minoxidil) and 5α-reductase inhibitors (e.g., finasteride and dutasteride), available for androgenetic alopecia, these medications must be administered long-term (life-long) and induce various side effects including unwanted facial/body hair growth, dizziness, cardiac arrhythmias, fainting, chest pain, edema of hands/feet, unusual weight gain, fatigue, dyspnea (for potassium channel openers), sexual problems (such as decreased sexual interest/ability and decrease in the amount of semen/sperm released during sex), impotence (trouble getting or keeping an erection), testicle pain or swelling, increased breast size, and breast tenderness (for 5α-reductase inhibitors). Further, currently existing therapies fail to improve hair growth in patients having other causes of alopecia, such as alopecia areata, which affects 1.7% of people in the United States (approximately 4.6 million individuals).

There is a need for new approaches to prevent and treat hair loss.

### SUMMARY OF THE INVENTION

The invention provides methods of preventing or treating alopecia in a subject (e.g., a human subject), the methods including administering a plasmacytoid dendritic cell (pDC) to the skin of a subject (e.g., beneath the surface of the skin or topically). In all instances of the mention of a pDC herein, the mention should be considered to indicate one or more pDCs.

In various embodiments, the subject has or is at risk of developing androgenetic alopecia (e.g., male-pattern alopecia or female-pattern alopecia), drug-induced alopecia (e.g., chemotherapy-induced alopecia), infection-induced alopecia (e.g., infection by tinea capitis or kerion), alopecia caused by a systemic disorder (e.g., disorders that cause high fever, systemic lupus erythematosus, endocrine disorders, and nutritional deficiencies), alopecia areata, trauma-induced alopecia (e.g., trichotillomania, traction alopecia, central centrifugal cicatricial alopecia, burns, radiation, and pressure-induced (e.g., post-operative) hair loss), a primary hair shaft abnormality, autoimmune disease-induced alopecia, heavy metal poisoning-induced alopecia, or dermatologic condition-induced alopecia (e.g., dissecting cellulitis of the scalp). In various embodiments, the alopecia treated according to the methods of the invention excludes alopecia areata.

In various embodiments, the plasmacytoid dendritic cell is obtained from the subject to whom it is administered. In other embodiments, the plasmacytoid dendritic cell is obtained from a different individual of the same species or a different species from the subject to whom it is administered.

In various embodiments, the plasmacytoid dendritic cell is administered in combination with a TLR7, TLR7/8, and/or TLR9 agonist, and/or is cultured with a TLR7, TLR7/8, and/or TLR9 agonist prior to administration. The TLR7 agonist can optionally be selected from the group consisting of Imiquimod (R837), Imiquimod VacciGrade™, Resiquimod (R848), R848 VacciGrade™, Gardiquimod™, Gardiquimod VacciGrade™, Loxoribine, Poly(dT), Vesatolimod (GS-9620), GS-986, CL264, CL307, TL8-506, Bropirimine, 7-Allyl-7,8-dihydro-8-oxoguanosine, and CU-CPT9a. The TLR9 agonist can optionally be a CpG oligonucleotide (e.g., a CpG oligonucleotide selected from the group consisting of CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10).

The invention further provides compositions including a plasmacytoid dendritic cell and a pharmaceutically acceptable carrier or diluent (e.g., phosphate buffered saline). In addition, the invention provides kits including such compositions and a syringe or applicator for administration of the composition and/or a second therapeutic agent. In various embodiments, the second therapeutic agent is selected from the group consisting of a TLR7 agonist, a TLR7/8 agonist, and a TLR9 agonist. The TLR7 agonist can optionally be selected from the group consisting of Imiquimod (R837), Imiquimod VacciGrade™′ Resiquimod (R848), R848 VacciGrade™, Gardiquimod™, Gardiquimod VacciGrade™, Loxoribine, Poly(dT), Vesatolimod (GS-9620), GS-986, CL264, CL307, TL8-506, Bropirimine, 7-Allyl-7,8-dihydro-8-oxoguanosine, and CU-CPT9a. The TLR9 agonist can optionally be a CpG oligonucleotide (e.g., a CpG oligonucleotide selected from the group consisting of CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10). In other embodiments, the second therapeutic agent is a potassium channel opener (e.g., minoxidol) or a 5α-reductase inhibitor (e.g., finasteride and dutasteride).

The invention additionally includes the use of the compositions and cells described herein for the methods described herein or in the preparation of medicaments for the purposes described herein.

The invention provides several advantages including, e.g., the non-invasive nature of adoptive cell transfer, the possibility of using autologous cells, the long term survival of cells and resulting long term effects of therapy (in comparison to usual medications that need to be applied multiple times per day), benefits of cell-based therapy, minimum pain, and fast recovery. In addition, the invention can be used in the treatment of patients with different types of alopecia.

Other features and advantages of the invention will be apparent from the following detailed description, the drawings, and the claims.

### BRIEF DESCRIPTION OF THE DRAWINGS

FIGS. 1A-1C are graphs showing the levels of TNF-α (FIG. 1A) and IL-6 (FIGS. 1B and 1C) induced by treatment with pDCs, which were treated with a TLR7 or TLR9 agonist, as indicated.

FIG. 2 is a series of photographs showing the results of treating mice with splenic pDCs, as compared to a control (PBS).

### DETAILED DESCRIPTION

The invention provides methods and compositions for use in preventing or treating hair loss or alopecia. The methods of the invention include the administration of plasmacytoid dendritic cells (pDCs) to induce or enhance hair growth. Optionally, the pDC therapy can be combined with other treatments (e.g., TLR7, TLR7/8, and/or TLR9 agonists; also see below). The methods and compositions of the invention are described further, as follows.

### Identification of Subjects

Subjects that can be treated using the methods and compositions of the invention include those suffering from, or at risk for, hair loss or alopecia. The subjects include human patients who have or are at risk of developing hair loss, e.g., as described herein. The subjects include adults, children, men, and women. The invention can also be used in the veterinary setting, e.g., with pets including cats and dogs, as well as livestock animals.

The methods and compositions of the invention can be used in the prevention and treatment of different causes of alopecia including, e.g., androgenetic alopecia (male-pattern or female-pattern hair loss); drugs (including chemotherapeutic agents); infection (e.g., tinea capitis and kerion); systemic disorders (e.g., disorders that cause high fever, systemic lupus erythematosus, endocrine disorders, and nutritional deficiencies); alopecia areata; trauma (including trichotillomania, traction alopecia, central centrifugal cicatricial alopecia, burns, radiation, and pressure-induced (e.g., postoperative) hair loss); primary hair shaft abnormalities; autoimmune diseases; heavy metal poisoning; and rare dermatologic conditions (e.g., dissecting cellulitis of the scalp). The methods and compositions of the invention can be used to prevent or treat scarring and non-scarring conditions, as are known in the art.

Plasmacytoid Dendritic Cells (pDCs)

The cells used in methods and compositions of the invention are plasmacytoid dendritic cells (pDCs), which circulate in the blood and can also be found in peripheral lymphoid organs. pDCs are bone marrow-derived innate immune cells that express Toll-like receptors (TLR) 7 and 9. In mice they express low levels of CD11c, which differentiates them from conventional dendritic cells (cDCs), and exhibit PDCA-1, Siglec-H, and CD45R/B220. In humans, pDCs are positive for blood-derived dendritic cell antigen (BDCA)-2 (CD303), BDCA-4 (CD304), and CD123. Upon activation, they produce large amounts of type 1 interferons (see, e.g., Tversky et al., Clin. Exp. Allergy 38(5):781-788, 2008; Asselin-Paturel et al., Nat. Immunol. 2(12):1144-1150, 2001; Nakano et al., J. Exp. Med. 194(8):1171-1178, 2001; Bjorck, Blood 98(13):3520-3526, 2001).

pDCs for use in the invention can be isolated from a subject to whom they are to be administered or they can be obtained from a donor (e.g., a human donor). pDCs can be isolated from blood or bone marrow using standard techniques including, e.g., density gradient centrifugation and marker-based cell separation. Optionally, the pDCs can be cultured and/or frozen prior to use. Furthermore, the pDCs can be obtained by the stimulation of cultured bone marrow cells. For example, peripheral blood mononuclear cells (PBMCs) can be isolated from blood using, e.g., Ficoll gradient density centrifugation. Then, pDCs can be isolated from PBMCs based on a pDC-specific or pDC-enriched marker (e.g. BDCA-2, BDCA-4, or CD123). An antibody against such a marker (e.g., an anti-BDCA-2, anti-BDCA-4, or anti-CD123 antibody) can be used in this isolation step using standard methods (e.g., microbead or magnetic bead-based separation or fluorescence-activated cell sorting [FACS]).

In a specific example, 5-10 mL blood is collected from a subject via routine venipuncture and is placed in a tube containing citrate as an anti-coagulant. Next, PBMCs are separated by standard Ficoll density gradient centrifugation. After isolating PBMCs, pDCs are selected via commercially available magnetic beads according to the manufacturer's instructions (Miltenyi Biotec). In brief, PBMCs are blocked with an anti-Fc receptor antibody for 15 minutes at room temperature (RT). Next, samples are labeled by incubation with an anti-BDCA2 antibody conjugated with microbeads for 30 minutes at 4° C. Cells labeled with magnetic bead-conjugated BDCA-2 antibodies (which will constitute pDCs) are then applied to a separation column, placed in a separation device standing on a magnetic field. By washing the separation column with sterile washing buffer, BDCA2-negative cells (non-pDCs) are washed out, while BDCA-2⁺ labeled pDCs stay attached to the column. At this step, the separation column is removed from the magnetic field and pDCs are eluted by pushing washing buffer through the column. After separation, the number of pDCs is determined by routine Trypan blue staining on a portion of collected cells. The purity of the sample is measured by immunofluorescence staining with a BDCA2 fluorochrome-conjugated antibody (as well as antibodies against other human pDC markers including, e.g., BDCA-4 and CD123, if needed) and analysis with FACS. In case the analysis shows unsatisfactory purity of the isolated cells (e.g., less than 85%), purity can be improved by another round of magnetic separation. Cells are then centrifuged and resuspended in sterile saline for adoptive transfer purposes.

In various embodiments, the pDCs are optionally cultured in the presence of a TLR7, TLR7/8, and/or TLR9 agonist prior to administration. Examples of these agents are provided below. In various embodiments, the pDCs are thus optionally cultured in the presence of 0.1-100 μg/mL (e.g., 0.5, 1, 5, 10, 25, or 50 μg/mL or at any amount between any ranges between these amounts) of a TLR7, TLR7/8, and/or TLR9 agonist for 0.5 hours to 5 days (e.g., 1, 2, 4, 8, 12, 18, or 24 hours, or 2, 4, or 5 days, or any length of time between any ranges between these lengths of time). In other embodiments, the pDCs are stimulated with a cytokine or genetically modified prior to use.

In addition to pDCs, medium in which pDCs are cultured (e.g., cell-free culture medium) can also be used in the methods and compositions of the invention, as such medium includes therapeutic molecules that are secreted from pDCs.

### Compositions

The invention also includes compositions including pDCs as described herein, for use in, e.g., the methods described herein. Such compositions each include pDCs and a pharmaceutically acceptable carrier or diluent. For example, pDCs prepared, e.g., as described above, can be diluted or concentrated to a final concentration of, e.g., about 10⁴-10⁸, 10⁵-10⁷, or 10⁶ cells per mL in a pharmaceutically acceptable carrier or diluent. The desired concentration of cells will vary depending on the method of administration and the type and severity of the disease or condition being treated. Depending upon the particular application, the carrier or diluent can be selected from, e.g., liquids, creams, drops, or ointments, as can be determined by those of skill in the art. For example, a solution may be used (e.g., phosphate buffered saline, sterile saline, or sterile culture medium (e.g., RPMI or DMEM)). The cells may further be administered in the cell culture medium in which they were cultured. The compositions used in the invention typically include pDCs, which are at least 50% (e.g., at least 60%, 75%, 90%, 95%, 99%, or 100%) of the cells present in the compositions.

Other compositions of the invention include cell culture medium (e.g., cell-free culture medium) in which pDCs have been cultured, and thus which contains therapeutic molecules that are secreted from pDCs.

### Methods of Treatment

pDCs or culture medium, as described above, can be administered to a subject to be treated according to the methods of the invention using methods that are known in the art for administration to or beneath the skin (e.g., the skin of the scalp). Thus, the methods of the invention include administration by injection under the skin (e.g., by subcutaneous or intradermal injection). The invention also includes administration involving the use of mechanical or chemical abrasion of the skin. In addition, the invention includes the use of topical administration. The methods of the invention typically involve administration to an area of the skin where there are hair follicles, and where it is desired to induce, increase, or maintain hair growth. The pDCs can be administered in amounts including, e.g., 10⁴-10⁸, 10⁵-10⁷, or 10⁶ cells per mL, as noted above.

Treatment according to the methods of the invention can be carried out using regimens that are determined to be appropriate by those of skill in the art based on factors including, for example, the type of disease, the severity of disease, the results to be achieved, and the age and general health of the patient. Treatment according to the methods of the invention thus can take place just once, or can be repeated (e.g., 2, 3, 4, 5, 6, 7, 8, 9, 10, or more times). In the case of multiple treatments, appropriate intervals between treatments can be selected by those of skill in the art. The invention thus includes, e.g., hourly, daily, weekly, monthly, bi-monthly, semi-annual, or annual treatments.

Adoptive transfer of pDCs or culture medium, as described above, can be used to prevent or treat alopecia to achieve a reduction in hair loss (or an increase in hair growth) of, for example, 10% or more (e.g., at least 10%, 20%, 30%, 40%, 50%, 60%, 70%, 80%, 90%, or 100%), as compared to the amount of hair loss (or hair growth) obtained in an untreated individual. For example, hair loss can be reduced by 25%, 50%, 2-fold, 5-fold, 10-fold or more, or is eliminated. Alternatively, hair growth can be increased by 25%, 50%, 2-fold, 5-fold, 10-fold or more.

In the case of prophylactic treatment, subjects at risk of developing alopecia due to, e.g., a disease or condition described herein, can be treated prior to hair loss or when hair loss first appears, to prevent development or worsening of hair loss. For example, in subjects already presenting with hair loss, further hair loss can be prevented by the methods of the present invention.

### Combinations

Administration of pDCs according to the invention can be done in combination with one or more other agents. For example, the pDCs can be administered in combination with one or more TLR7, TLR7/8, or TLR9 agonist. The pDCs can optionally be administered at substantially the same time as the agonist(s). For example, they can be co-administered in the same composition as one another, or they can be administered in separate compositions but within a few minutes of one another. Alternatively, the agonist(s) can be administered before or after the pDCs, according to the invention. They can thus be administered during the same treatment appointment (within, e.g., 5-120, 10-60, or 15-30 minutes of one another) or on separate days (e.g., 1, 2, 3, 4, 5, 6, or 7 days apart, or 1, 2, 3, 4, or more weeks apart).

TLR7 agonists that can be used in the invention include, e.g., Imiquimod (R837), Imiquimod VacciGrade™, Resiquimod (R848), R848 VacciGrade™, Gardiquimod™, Gardiquimod VacciGrade™′ Loxoribine, Poly(dT), Vesatolimod (GS-9620), GS-986, CL264, CL307, TL8-506, Bropirimine, 7-Allyl-7,8-dihydro-8-oxoguanosine, and CU-CPT9a (see, e.g., InvivoGen and Sigma Aldrich). As is understood in the art, some TLR7 agonists (e.g., Gardiquimod, Imiquimod, and Resiquimod) are also TLR8 agonists. Use of such dual agonists (e.g., TLR7/8 agonists) are also included within the scope of the invention.

TLR9 agonists that can be used in the invention include CpG oligonucleotides. In specific non-limiting examples, the following agents can be used: CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

The agonists can be administered in amounts determined to be appropriate by those of skill in the art. Exemplary amounts of TLR7 (or TLR7/8) agonists for administration are one or more drops applied beneath the skin or topically (e.g., 1, 2, 3, 4, or 5 drops) of a 0.05-10% w/v (e.g., 0.1-8%, 1-6%, 2-5%, or 3-4% w/v) solution, while exemplary amounts of TLR9 agonists are 0.5-100 mg (e.g., 1-75, 3-50, 5-40, 10-30, or 15-25 mg) per dose. Optionally, the TLR7 (including TLR7/8 agonists) and TLR9 agonists are comprised within pharmaceutically acceptable compositions, such as a liquid, cream, or gel for use in topical administration, as known in the art. The agonists are included within these compositions in amounts sufficient to provide a desired dosage, using a desired volume, as can be determined by those of skill in the art.

The pDC therapy of the invention (with or without the TLR-based therapy noted above) can optionally be carried out in combination with known methods for the treatment of alopecia including, e.g., the administration of potassium channel openers (e.g., minoxidil) and 5α-reductase inhibitors (e.g., Finasteride and Dutasteride).

### Kits

The invention also provides kits that include pDCs (e.g., pDCs present in a pharmaceutically acceptable carrier or diluent) for use in preventing or treating alopecia, such as alopecia related to one or more of the diseases or conditions described herein. The kits can optionally include an agent or device for delivering pDCs to or through the skin. For example, the kits may optionally include agents or devices for permeabilizing the skin (e.g., a tool for mechanically disrupting the skin). In other examples, the kits may include one or more sterile syringes or needles. Further, the kits may optionally include other agents, for example, anesthetics, antibiotics, or other agents for use in preventing or treating alopecia (e.g., the other therapeutic agents described herein).

The following non-limiting examples are illustrative of the present disclosure.

### EXAMPLES

### Example 1

In order to evaluate whether adoptive transfer of pDCs may cause an adverse immune response, we measured the levels of IL-6 and TNF-α, two pro-inflammatory cytokines in the skin. In brief, we sorted splenic pDCs and cultured them in vitro for 24 hours under treatment with PBS control, TLR7 agonist, or TLR9 agonist. Then we performed local adoptive transfer of pDCs by subcutaneous injections of in vitro treated pDCs or a media only control. 14 days later, we measured the protein levels of the aforementioned cytokines. Our findings show that local adoptive transfer of pDCs is safe, as it is not accompanied with increased levels of pro-inflammatory cytokines.

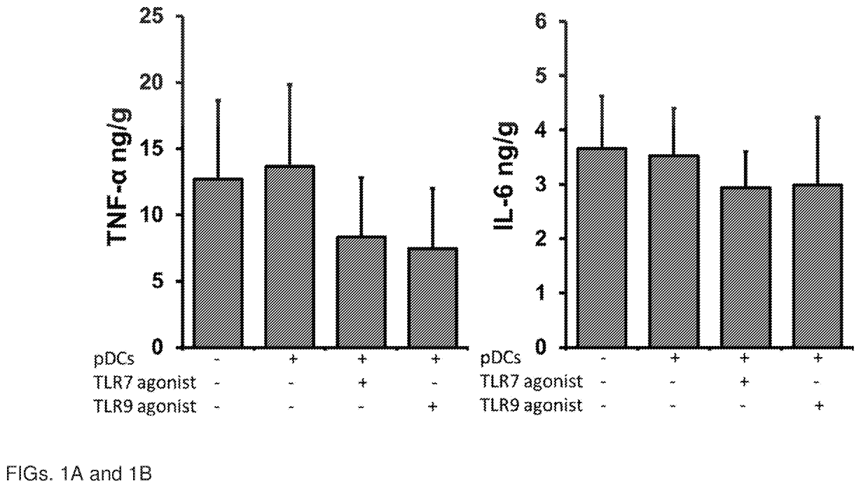

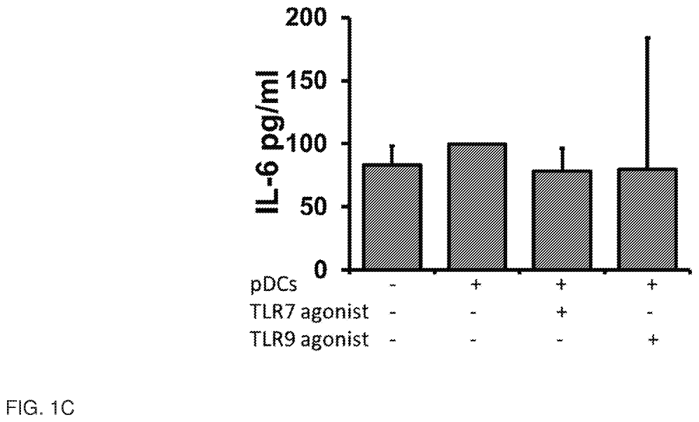

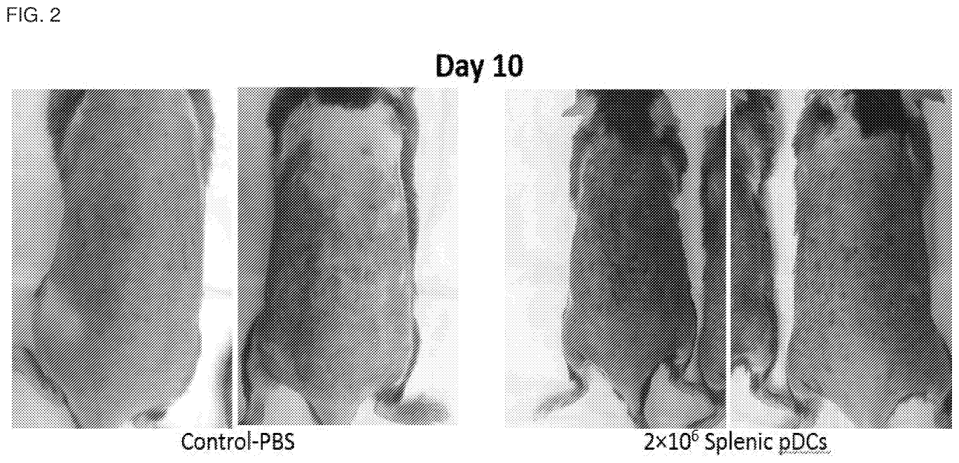

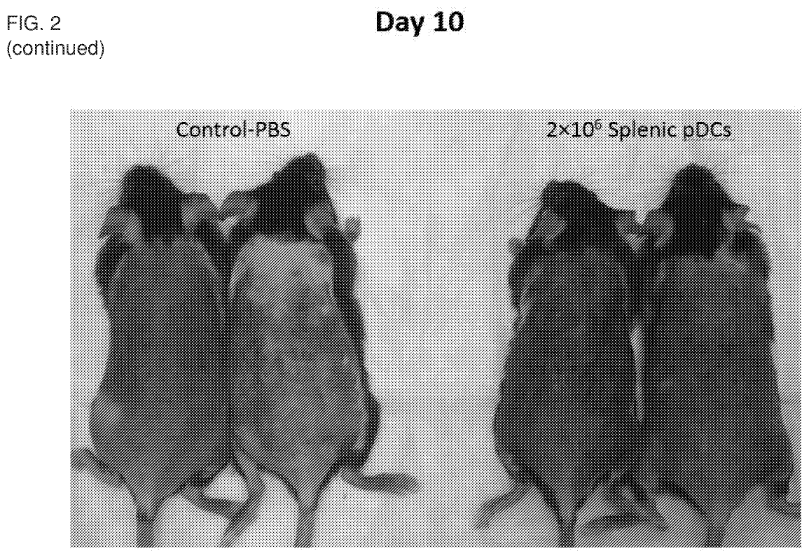

In more detail, splenic pDCs were sorted and cultured in vitro for 24 hours under treatment with PBS control, 1 μg/mL imiquimod (TLR7 agonist; InvivoGen, San Diego, Calif.), or 1 μg/mL phosphorothioate CpG 1826 oligonucleotide (CpG-ODN; a synthetic specific TLR-9 agonist; InvivoGen). Hair was removed from the backs of wildtype C57BL/6 mice using a clipper, followed by application of hair removal lotion (Nair, Church & Dwight, Township, N.J.). Next, 2×10⁶ cells or control media was subcutaneously injected into the backs of the mice. 14 days later, mice were euthanized and skin was sampled using a 4 mm skin biopsy punch. For protein extraction, initially skin samples were incubated for 30 minutes in ice-cold RIPA buffer containing 1 mM phenylmethylsulfonyl fluoride (PMSF; Sigma-Aldrich) and 30 μg/mL aprotinin (Sigma-Aldrich) at 4° C. Next, samples were homogenized using a Branson sonifier (Branson Ultrasonics, Danbury, Conn.) and were further incubated for 30 minutes at 4° C. The homogenate was centrifuged at 15,000 g for 20 minutes at 4° C., and the supernatant was collected and total protein levels were measured using DC™ protein assay (Bio-Rad Laboratories, Hercules, Calif.). Levels of IL-6 and TNF-α were measured via a customized Bioplex multiplex mouse cytokine assay (Bio-Rad Laboratories). As shown in FIGS. 1A and 1B, local adoptive transfer of pDCs does not alter local levels of TNF-α (p>0.05; FIG. 1A) or IL-6 (p>0.05; FIG. 1B). The p values for the comparisons are as follows: for TNF-α: p=0.44, for IL-6: p=0.73. Additional results testing IL-6 levels are provided in FIG. 10.

### Example 2

Mice were treated with pDCs, as described as follows. On day 0, 5 wild type C57BL/6 mice (male, aged 8 weeks) were anesthetized using ketamine/xylazine and their back hairs were removed by initial shaving using clippers followed by application of Nair hair removal solution. Starting on day 1, mice received 1 mL PBS (control) (n=2), or 2×10⁶ GFP* pDCs (sorted from the spleen of DPE-GFP×RAG1^{-/-} mouse) (n=2) by means of subcutaneous injection. Mice were imaged on Day 10. Treatment of mice with pDCs results in increased hair growth, as compared to the PBS control.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

All publications and patent applications mentioned in this specification are herein incorporated by reference to the same extent as if each independent publication or patent application was specifically and individually indicated as being incorporated by reference in their entirety.

Use of singular forms herein, such as “a” and “the,” does not exclude indication of the corresponding plural form, unless the context indicates to the contrary. Similarly, use of plural terms does not exclude indication of a corresponding singular form.

Some embodiments are within the scope of the following numbered paragraphs.

1. A method of preventing or treating alopecia in a subject, the method comprising administering a plasmacytoid dendritic cell (pDC) to or beneath the skin of a subject.

2. The method of paragraph 1, wherein the pDC is delivered beneath the surface of the skin.

3. The method of paragraph 1 or 2, wherein the pDC is delivered topically.

4. The method of any one of paragraphs 1 to 3, wherein the subject has or is at risk of developing androgenetic alopecia, drug-induced alopecia, infection-induced alopecia, alopecia caused by a systemic disorder, alopecia areata, trauma-induced alopecia, a primary hair shaft abnormality, autoimmune disease-induced alopecia, heavy metal poisoning-induced alopecia, or dermatologic condition-induced alopecia.

5. The method of any one of paragraphs 1 to 4, wherein the alopecia is androgenetic alopecia.

6. The method of paragraph 4 or 5, wherein the androgenetic alopecia is male-pattern alopecia.

7. The method of paragraph 4 or 5, wherein the androgenetic alopecia is female-pattern alopecia.

8. The method of any one of paragraphs 1 to 4, wherein the alopecia is drug-induced alopecia.

9. The method of paragraph 4 or 8, wherein the drug-induced alopecia is chemotherapy-induced alopecia.

10. The method of any one of paragraphs 1 to 4, wherein the alopecia is due to an infection.

11. The method of paragraph 4 or 10, wherein the infection is tinea capitis or kerion.

12. The method of any one of paragraphs 1 to 4, wherein the alopecia is due to a systemic disorder.

13. The method of paragraph 4 or 12, wherein the systemic disorder is selected from disorders that cause high fever, systemic lupus erythematosus, endocrine disorders, and nutritional deficiencies.

14. The method of any one of paragraphs 1 to 4, wherein the alopecia is alopecia areata.

15. The method of any one of paragraphs 1 to 4, wherein the alopecia is due to trauma.

16. The method of paragraph 4 or 15, wherein the trauma is selected from trichotillomania, traction alopecia, central centrifugal cicatricial alopecia, burns, radiation, and pressure-induced hair loss.

17. The method of any one of paragraphs 1 to 4, wherein the alopecia is due to primary hair shaft abnormalities.

18. The method of any one of paragraphs 1 to 4, wherein the alopecia is due to an autoimmune disease.

19. The method of any one of paragraphs 1 to 4, wherein the alopecia is due to heavy metal poisoning.

20. The method of any one of paragraphs 1 to 19, wherein the subject is a human subject.

21. The method of any one of paragraphs 1 to 20, wherein the plasmacytoid dendritic cell is obtained from the subject to whom it is administered.

22. The method of any one of paragraphs 1 to 20, wherein the plasmacytoid dendritic cell is obtained from an individual and/or species different from the subject to whom it is administered.

23. The method of any one of paragraphs 1 to 22, wherein the plasmacytoid dendritic cell is administered in combination with a TLR7, TLR7/8, and/or TLR9 agonist.

24. The method of any one of paragraphs 1 to 23, wherein the plasmacytoid dendritic cell is cultured with a TLR7, TLR7/8, and/or TLR9 agonist prior to administration.

25. The method of paragraph 23 or 24, wherein the TLR7 agonist is selected from the group consisting of Imiquimod (R837), Imiquimod VacciGrade™, Resiquimod (R848), R848 VacciGrade™′ Gardiquimod™, Gardiquimod VacciGrade™, Loxoribine, Poly(dT), Vesatolimod (GS-9620), GS-986, CL264, CL307, TL8-506, Bropirimine, 7-Allyl-7,8-dihydro-8-oxoguanosine, and CU-CPT9a.

26. The method of any one of paragraphs 23 to 25, wherein the TLR9 agonist is a CpG oligonucleotide.

27. The method of paragraph 26, wherein the CpG oligonucleotide is selected from the group consisting of CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

28. A composition comprising a plasmacytoid dendritic cell and a pharmaceutically acceptable carrier or diluent.

29. The composition of paragraph 28, wherein the pharmaceutically acceptable diluent is phosphate buffered saline.

30. A kit comprising the composition of paragraph 28 or 29 and a syringe or applicator for administration of said composition and/or a second therapeutic agent.

31. The kit of any one of paragraphs 28 to 30, wherein the second therapeutic agent is selected from the group consisting of a TLR7 agonist, a TLR7/8 agonist, and a TLR9 agonist.

32. The kit of paragraph 31, wherein the second therapeutic agent is a TLR7 agonist selected from the group consisting of Imiquimod (R837), Imiquimod VacciGrade™, Resiquimod (R848), R848 VacciGrade™, Gardiquimod™, Gardiquimod VacciGrade™, Loxoribine, Poly(dT), Vesatolimod (GS-9620), GS-986, CL264, CL307, TL8-506, Bropirimine, 7-Allyl-7,8-dihydro-8-oxoguanosine, and CU-CPT9a.

33. The kit of paragraph 31, wherein the second therapeutic agent is a TLR9 agonist, which is a CpG oligonucleotide.

34. The kit of paragraph 33, wherein the CpG oligonucleotide is selected from the group consisting of CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

35. The kit of any one of paragraphs 30 to 34, wherein the second therapeutic agent is a potassium channel opener or a 5α-reductase inhibitor.

36. The kit of paragraph 35, wherein the second therapeutic agent is a potassium channel opener, which is minoxidol.

37. The kit of paragraph 30, wherein the second therapeutic agent is a 5α-reductase inhibitor selected from finasteride and dutasteride.

Other embodiments are within the scope of the following claims.

What is claimed is:

1. A method of preventing or treating alopecia in a subject, the method comprising administering a plasmacytoid dendritic cell (pDC) to the skin of the subject.

2. The method of claim 1, wherein the pDC is delivered beneath the surface of the skin.

3. The method of claim 2, wherein the pDC is delivered topically.

4. The method of claim 1, wherein the subject has or is at risk of developing androgenetic alopecia, drug-induced alopecia, infection-induced alopecia, alopecia caused by a systemic disorder, alopecia areata, trauma-induced alopecia, a primary hair shaft abnormality, autoimmune disease-induced alopecia, heavy metal poisoning-induced alopecia, or dermatologic condition-induced alopecia.

5. The method of claim 4, wherein the alopecia is androgenetic alopecia.

6. The method of claim 4, wherein the androgenetic alopecia is male-pattern alopecia.

7. The method of claim 4, wherein the androgenetic alopecia is female-pattern alopecia.

8. The method of claim 4, wherein the alopecia is drug-induced alopecia.

9. The method of claim 4, wherein the drug-induced alopecia is chemotherapy-induced alopecia.

10. The method of claim 4, wherein the alopecia is due to an infection.

11. The method of claim 4, wherein the infection is tinea capitis or kerion.

12. The method of claim 4, wherein the alopecia is due to a systemic disorder.

13. The method of claim 4, wherein the systemic disorder is selected from disorders that cause high fever, systemic lupus erythematosus, endocrine disorders, and nutritional deficiencies.

14. The method of claim 4, wherein the alopecia is alopecia areata.

15. The method of claim 4, wherein the alopecia is due to trauma.

16. The method of claim 4, wherein the trauma is selected from trichotillomania, traction alopecia, central centrifugal cicatricial alopecia, burns, radiation, and pressure-induced hair loss.

17. The method of claim 4, wherein the alopecia is due to primary hair shaft abnormalities.

18. The method of claim 4, wherein the alopecia is due to an autoimmune disease.

19. The method of claim 4, wherein the alopecia is due to heavy metal poisoning.

20. The method of claim 1, wherein the subject is a human subject.

21. The method of claim 1, wherein the plasmacytoid dendritic cell is obtained from the subject to whom it is administered.

22. The method of claim 1, wherein the plasmacytoid dendritic cell is obtained from an individual and/or species different from the subject to whom it is administered.

23. The method of claim 1, wherein the plasmacytoid dendritic cell is administered in combination with a TLR7, TLR7/8, and/or TLR9 agonist.

24. The method of claim 1, wherein the plasmacytoid dendritic cell is cultured with a TLR7, TLR7/8, and/or TLR9 agonist prior to administration.

25. The method of claim 23, wherein the TLR7 agonist is selected from the group consisting of Imiquimod (R837), Imiquimod VacciGrade™, Resiquimod (R848), R848 VacciGrade™′ Gardiquimod™, Gardiquimod VacciGrade™, Loxoribine, Poly(dT), Vesatolimod (GS-9620), GS-986, CL264, CL307, TL8-506, Bropirimine, 7-Allyl-7,8-dihydro-8-oxoguanosine, and CU-CPT9a.

26. The method of claim 23, wherein the TLR9 agonist is a CpG oligonucleotide.

27. The method of claim 26, wherein the CpG oligonucleotide is selected from the group consisting of CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

28. A composition comprising a plasmacytoid dendritic cell and a pharmaceutically acceptable carrier or diluent.

29. The composition of claim 28, wherein the pharmaceutically acceptable diluent is phosphate buffered saline.

30. A kit comprising the composition of claim 28 and a syringe or applicator for administration of said composition and/or a second therapeutic agent.

31. The kit of claim 30, wherein the second therapeutic agent is selected from the group consisting of a TLR7 agonist, a TLR7/8 agonist, and a TLR9 agonist.

32. The kit of claim 31, wherein the second therapeutic agent is a TLR7 agonist selected from the group consisting of Imiquimod (R837), Imiquimod VacciGrade™, Resiquimod (R848), R848 VacciGrade™, Gardiquimod™, Gardiquimod VacciGrade™, Loxoribine, Poly(dT), Vesatolimod (GS-9620), GS-986, CL264, CL307, TL8-506, Bropirimine, 7-Allyl-7,8-dihydro-8-oxoguanosine, and CU-CPT9a.

33. The kit of claim 31, wherein the second therapeutic agent is a TLR9 agonist, which is a CpG oligonucleotide.

34. The kit of claim 33, wherein the CpG oligonucleotide is selected from the group consisting of CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

35. The kit of claim 30, wherein the second therapeutic agent is a potassium channel opener or a 5α-reductase inhibitor.

36. The kit of claim 35, wherein the second therapeutic agent is a potassium channel opener, which is minoxidol.

37. The kit of claim 30, wherein the second therapeutic agent is a 5α-reductase inhibitor selected from finasteride and dutasteride.
